(19) United States
(12) Patent Application Publication
(10) Pub. No.: US 2011/0117639 A1
(43) Pub. Date: May 19, 2011

(54) Taylor Vortex Flow Bioreactor for Cell Culture

(76) Inventors: Claudio Alberto Torres Suazo, Sao Carlos (BR); Roberto de Campos Giordano, Cartos (BR); Patricia Aparecida Santiago, Sao CArlos (BR)

(21) Appl. No.: 12/996,052

(22) PCT Filed: Jun. 5, 2009
(86) PCT No.: PCT/BR2009/000160

§ 371 Date: Jan. 27, 2011

(30) Foreign Application Priority Data
Jun. 5, 2008 (BR) PI0803630-6

(51) Int. Cl. C12M 1/10; C12M 3/00

(52) U.S. Cl. 435/302.1

(57) ABSTRACT

The invention concerns a rotating wall vessel bioreactor (100) using Taylor vortex flow for cell culture in the annular space between the two concentric cylindrical bodies, wherein the internal one (2) is rotating and the external one (1) is stationary. The internal rotating body (2) is composed of an external wall around which a polymeric tubular membrane (6) is wrapped, which is connected to the hollow tubular axis (5) of the internal body (2) for the introduction of gases into the annular space (d). Said annular space (d) is filled with a suspension of cells or cells immobilized on microcarriers.

### FIELD OF THE INVENTION

The present invention relates to the field of bioreactors used for the culture of animal and plant cells, more specifically, to a bioreactor based on Taylor vortexes flow.

### BACKGROUND OF THE INVENTION

The containment of a fluid between two concentric cylinders, which the internal (and maybe the external) cylinder is rotating, is a classical theme in fluid dynamics and it was described for the first time by Taylor in the work Taylor, G. I. “Stability of a viscous liquid between rotating cylinders”. Philosophical Transactions of Royal Society A, v. 223, p. 289-343, 1923.

In his studies, the researcher examined the beginning of the formation of a secondary flow in the annular space between two concentric cylinders under rotation simultaneous or in separate. Taylor proved experimentally that in internal cylinder rotation speeds below of a determined value, the fluid simply moved tangentially in the annular space. However, in the moment the rotation speed exceeded this limit, the movement, before tangential (Couette main flow), was superimposed by a helicoidal trajectory in several layers and with alternated directions of rotation. This pattern of flow was denominated “vortexes flow”. Another result observed was that the rotation of solely the external cylinder, while the internal remains stationary, does not enable the formation of vortexes.

Taylor, through theorical studies, disregarded the non-linear terms of the Navier-Stokes equations and resolved the equations for the perturbations of the basic flow (Couette) using series of Bessel-Fourier. This way, Taylor could calculate the minimum conditions for the vortexes establishment and amplify the previous analysis of stability proposed by Rayleigh, in 1916, expanding it to rotational flows and uncompressible viscous fluids. Another result obtained by Taylor was the possibility of determining the size of the vortexes and their rotation direction, once, essentially, the amplitude of the secondary flow is equal the double of the annular space. Therefore, each pair of vortexes spinning in alternate directions constitute an unit that reproduce itself in an stable manner throughout the whole annular space, and each vortex individually is contained in a approximately square section region, with height equals to the width of the annular space.

From these results, the researchers begin to denominate as Taylor vortexes flow or Taylor-Couette flow the secondary flow produced when an internal cylinder spins while the external one remains stationary. The reference of the researchers to Couette is in regard to the device studied by Maurice Couette, in 1890, which was composed of two concentric cylinders, but, in this case, the internal cylinder remained static and the external one under rotation, according to Donnelly, R. J. “Taylor-Couette Flow: The Early Days”. American Institute of Physics. Physics Today. p. 3238, November 1991.

In the following years, publications about the Taylor vortexes flow were multiplied in the most diverse areas. Among them, studies involving the characterization of the flow regimes, according to Coles, D. “Transition in circular Couette flow” Journal of Fluid Mechanics, v. 21, n.3, p. 385-425, 1965 and Davey, A. “The growth of Taylor vortices in flow between rotating cylinders”. Journal of Fluid Mechanics, v. 14, p. 336-368. 1962, and the study of mass and heat transfer, according to the works: Kataoka, K. “Heat transfer in a Taylor vortex flow”. Journal of Chemical Engineering Japan, 8, 472-476, 1975; Legrand, J. et al. “Overall mass transfer to the rotating inner electrode of a concentric cylindrical reactor with axial flow”. Electrochimica Acta, 25, 669-673, 1980; Kataoka, K. et al. “Mass transfer in the annulus between two coaxial rotating cylinders”. Heat and Mass Transfer in Rotating Machinery (eds. Metzger, D. E. and Afagan, N. H.) 143-153, Hemisphere, New York, 1984; Legrand, J. and Coeuret, F. “Transfert de Matière Liquide-Paroi et Hydrodynamique de l'Écoulement de Couette-Taylor-Poiseuille Biphasique”. Can. J. Chem. Engine. 65, 237-243, 1987; Moore, C. M. V. “Characterization of a Taylor-Couette vortex flow reactor. 239 p. Thesis (Doctor of Philosophy in Chemical Engineering), Massachusetts Institute of Technology (MIT), United States of America. 1994; Desmet, G. et al. “Local and global dispersion effects in Couette-Taylor flow II: Quantitative measurements and discussion of reactor performance”. Chemical Engineering Science, v. 5, n. 8, p. 1299-1309. 1996; Wronski, S. et al. “Mass transfer in gas-liquid Couette-Taylor flow in membrane reactor”. Chemical Engineering Science, v. 54, p. 2963-2967. 1999; Giordano, R. C, et al. “Analysis of a Taylor-Poiseuille vortex flow reactor-I: Flow patterns and mass transfer characteristics”. Chemical Engineering Science, v. 53, n. 20, p. 3635-3652, 1998; Resende, M. M et al. “Estimation of mass transfer parameters in a Taylor-Couette-Poiseuille heterogeneous reactor”. Brazilian Journal of Chemical Engineering, v. 21, n. 02, p. 175-184, 2004.

As previously mentioned, publications directed to applications of Taylor vortexes flow increased considerately in number since the study performed by Taylor in 1923, wherein in the last two decades the employment of this type of flux was extended to bench bioreactors, according to Giordano, R. L. C. et al. “Analysis of a Taylor-Poiseuille vortex flow reactor II: reactor modeling and performance assessment using glucose-fructose isomerization as test reaction. Chemical Engineering Science, v. 55, p. 3611-3626. 2000; Dutta, P. K; Ray, A. K. “Experimental investigation of Taylor vortex photocatalytic reactor for water purification”. Chemical Engineering Science, vol. 59, p. 5249-5259. 2004.

In the '90s, based on the conception of concentric cylinders, bioreactors denominated RWVB—rotating wall vessel bioreactor—were developed by the North American Space Agency (NASA). These equipments are currently commercialized by the company Synthecon (Houston-USA) and has as objective obtain “microgravity” (absence of gravity), which means, to minimize the shear stress present in the fluid-dynamic environment of bioreactors during cellular cultivation, principally of animal and plant cells. In order to obtain this, these equipments are operated in the Couette flow regime, with rotation axis placed horizontally. In this case, the external cylinder is rotating, while the internal one is stationary or spins in the same rotation speed of the external. Another characteristics of the RWVBs is the oxygenation of the culture media, wherein is attained by a flat membrane fixed in the internal cylinder. These equipments do not present geometric characteristics and do not operate with the intention of forming the Taylor-Couette flow. Works regarding this type of bioreactor were published, such as: Unsworth, B. R. and Lelkes, P. I “Growing tissues in microgravity”. Nature Medicine. V. 4, n.8, p. 901-907. 1998; Cowger, N. L. et al. “Characterization of bimodal cell death of insect cells in a rotating-wall vessel and shaker flash”. Biotechnology and Bioengineering, v. 64, n. 1, p. 14-26. 1999; Sun, X and Linden, J. C. “Shear stress effects on plant cell suspension cultures in a rotating wall vessel bioreactor”. Journal of Industrial Microbiology & Biotechnology, v. 22, p. 44-47. 1999; Hammond, T. G. and Hammond J. M. Optimized suspension culture: the rotating-wall vessel”. AJP—Renal, v. 281, p. 12-25. 2001; O'Connor, K. C et al. “Prolonged shearing of insect cells in a Couette bioreactor” Enzyme and Microbial Technology, v. 31, p. 600-608. 2002; Saini, S. and Wick, T. M. “Concentric cylinder bioreactor for production of tissue engineered cartilage: effect of seeding density and hydrodynamic loading on construct development”. Biotechnology Progress, v. 19, p. 510-521, (2003); Klement, B. J. et al. “Skeletal tissue growth, differentiation and mineralization in the NASA Rotating Wall Vessel”. Bone, v. 34, p. 487-498. 2004; Liu, T et al. “Analysis on forces and movement of cultivated particles in a rotating wall vessel bioreactor”. Biochemical Engineering Journal, v. 18, p. 97-104 (2004); Martin, Y and Vermette, P. “Bioreactors for tissue mass culture: Design, characterization, and recent advances”. Biomaterials, v. 26, p. 7481-7503. 2005.

Other bioreactors of the RWVB type, but that operates in Taylor vortexes flow regime, are evaluated in Haut, B. et al. “Hydrodynamics and mass transfer in a Couette-Taylor bioreactor for the culture of animal cells”. Chemical Engineering Science, v. 58, p. 777-784. 2003; Curran, S. J. and Black, R. A. “Quantitative experimental study of shear stress and mixing in progressive flow regimes within annular-flow bioreactors” Chemical Engineering Science, v. 59, p. 5839-5868. 2004 e Curran, S. J. and Black, R. A. “Oxygen transport and cell viability in an annular flow bioreactor: comparison of laminar Couette and Taylor-Vortex flow regimes”. Biotechnology and Bioengineering, v. 89, n. 7, p. 766-774, Mar. 30, 2005. In these works, although innovations regarding the use of bioreactor for the cultivation of animal and plant cells are proposed, serious obstacles hinder the scaling up process. These limitations involve the absence of devices to promote mass and heat transfers as efficient as those of the bioreactor of the present invention. In the RWVBs, the supply of gases to the culture media can occur superficially in the gas-liquid interface or through the oxygenizer located externally. These oxygenation systems cause the restriction in the volumetric capacity (100 mL) of the equipment, once these techniques are not appropriated for the cultivations with high cellular density (>10⁶ cells·mL⁻¹). The limitation to the heat transport is due to the absence of heat exchange in the bioreactors structure.

All these restrictions are surpassed in the bioreactor of the invention, denominated as “Bioreactor of Taylor Vortexes Flow” (BTVF), as it will be seen further in the present document. The BTVF has efficient systems to the mass and heat transfer enabling scaling up.

The patent literature presents several documents regarding the subject.

The patent JP 07-117088 relates to a procedure of adherent animal cells cultivation employing and comparing the performance (principally regarding cell viability) between bioreactors of the agitation tank type and the concentric cylinders type. The patent in question focuses on a procedure for cell cultivation, and does not give any constructive or innovation conception detail of a bioreactor of Taylor vortexes flow. It is worth highlighting that the relevant advantage of the Taylor-Couette flow are the low shear stress that characterize it, and that they provide an environment more amenable to cell culture. Therefore, the objective of the patent JP 2752918 is solely to compare two agitation systems, which one of them is the conventional method, composed by impeller and present in bioreactor of the agitation tank type, and other by Taylor vortexes flow in bioreactor of concentric cylinders.

In the publication JP 2001-192215, the instrument and method employed for regenerating a protein is described. The equipment in question is composed by two concentric cylinders, wherein the internal one is rotating and the external one is stationary. The equipment is placed in order to spin horizontally. Between the internal and external cylinders there is a tube constituted of a membrane permeable to the flow of regenerated protein. It is in the annular space between the internal cylinder and the membrane that the Taylor vortexes flow is formed. The conception of the equipment and its biotechnological applications are different from the bioreactor present in the present application.

The patent GB2097817 cites a bioreactor of Taylor vortexes flow used for the cultivation of animal and plant cells. The bioreactor is formed by a chamber constituted of external cylinder, made of hollow steel in which a flat permeable membrane was placed. In the interior of the membrane pairs of concentric cylindrical tubes are assembled. The oxygenation of the culture media occurs through this membrane. With that, there is a limitation in the relation of oxygen exchange area by reactor volume. It is, therefore, a distinct conception of the one of the present invention.

In the patent U.S. Pat. No. 3,647,632 a perfusion bioreactor for cellular cultivation is described. The equipment is composed by a glass tank and in its interior it is found, close to the base, a rotating filter made of stainless steel screen responsible for retention of the cells in its interior. The bioreactor in question has a conception very distinct from the one of the present invention, since it does not enable the formation of Taylor vortexes.

As well as with the previous patent, U.S. Pat. No. 5,057,428 presents the description of bioreactor with a different conception from the one of the present invention. The document in question relates to a bioreactor of a non-potable type for the culture of animal and plant cells. The equipment is composed by two concentric cylinders, wherein the internal one is composed by a beam of tubes distributed and fixed by a spacing disk. The cells are contained in a cylindrical container made of hollow stainless steel and placed parallel throughout the whole internal cylinder, enabling the contact with the culture media and oxygen.

The published North American application U.S. 2006/0240544 described a bioreactor for cultivation of microorganisms, animal and plant cells. The equipment is composed by two concentric cylinders, wherein the internal one is rotating and divided in three compartments in order to separate culture media, cells and nutrient solutions. The bioreactor described in that document can be classified as perfusion bioreactor, which is different from the object of the present invention, since it does not adopt the conception of Taylor vortexes flow.

The patent U.S. Pat. No. 5,155,035 present a perfusion bioreactor based on the microgravity environment for cultivation of mammal's cells and a method for cultivation in this type of system. In the bioreactor, the culture media spin around the horizontal axis and the particles are kept suspended in the liquid in low shear stress. The document does not present as objective the project and construction of a bioreactor of Taylor vortexes flow.

Following the premise of procedure description, the publication WO 2005/007269 reports the methodology and instrument employed for the production of proteins. This publication uses a perfusion bioreactor for the cultivation of myeloma cells and, combined to the equipment, an external filtration system enables the separation of the expressed protein in the culture media. The invention described in that document present a different concept of the one of a bioreactor of Taylor vortexes flow.

In the patent U.S. Pat. No. 4,876,013 the method and several instruments to be used during a process of filtration, preferentially through the use of semi permeable membranes are described. This method and its several devices are employed in processes such as ultrafiltration, reverse osmosis, dialysis, pervaporation and microfiltration using the Taylor vortexes flow regime. The equipment is composed of two concentric cylinders, wherein the internal one is rotating through the use of an engine. The flat semi permeable membrane is localized to the wall of the internal cylinder and the material to be filtered is transported axially through the annular space. The mass transport can increase in one order of magnitude the filtration flux (i.e., the flow speed of the filtrate per filter area unit) regarding the conventional tangential filtration. Besides that, the vortexes flow is employed to aid in the maintenance of the disobstruction of the membrane surface during the continuous processes of filtration. The equipment described in the document U.S. Pat. No. 4,876,013 is employed in the process of filtration and not for cell culture.

The patent U.S. Pat. No. 5,968,355 relates to the construction of equipment based in the concept of Taylor vortexes flow and to its employment in the aseptic processing of pharmaceutical or biological materials, including collagen, gels and semi solids. The equipment has the function of filtrating and concentrating these materials. The equipment is composed of two concentric cylinders, wherein in the annular space it is found a semi permeable membrane responsible for separating substances. Once again, it is about an instrument for aseptic filtration and concentration of biological material and not about a bioreactor for the culture of animal and plant cells.

The patent U.S. Pat. No. 6,099,730 presents the description of an equipment and the methodology employed in the blood treatment. The instrument is composed of two concentric cylinders, wherein the internal one is rotating and the external one is stationary. In the annular space between the cylinders the Taylor vortexes flow is formed. In the external wall of the internal cylinder as well as in the internal wall of the external cylinder, the semi permeable membranes employed in the removal of blood substance that are considered toxic are localized. The equipment uses the principle of simultaneous separation and reaction inside it with the objective of increasing the efficiency of the blood detoxification or purification process without damaging the cells present within. As can be evaluated, the equipment in question, even applying the Taylor vortexes flow, is not used for animal cells cultivation but for a clinical treatment.

As can be observed, there are several articles and patents that employ the Taylor vortexes flow in biotechnological processes. However, when analyzing the present references in the literature, it was not found bioreactor with the characteristics described in the present application.

The need for human viral vaccines in the '50s, especially against poliomyelitis, propelled the bioprocesses in large scale of animal cells, because it was the first process to be performed in industrially, according to Griffiths, J. B. “Animal cell products, overview” in: Spier, R. E. (Ed.) Encyclopedia of cell technology, New York: John Willey & Sons, v. 1, p. 71-76. 2000.

The in last 20 years, it has been observed a fast increase in number and demand for biopharmaceutical products produced in processes involving the culture of animal cells. Currently, there are more than 30 licensed products, wherein the great part is recombinant proteins. This increase is due to principally the proved efficiency in the obtaintion of therapeutic compounds according to Butler, M. “Animal cell cultures: recent achievements and perspectives in the production of biopharmaceuticals”. Applied Microbiology and Biotechnology, v. 68, p. 283-291, 2005.

With the increasing development of products derived of cellular cultures, the necessity of development and optimization of the processes of production became evident.

The first bioreactors used for the cultivation of animal cells were derived from fermentators developed for the production of microorganisms, with little or no modification in their structure. This characteristic made them inappropriate for cell culture due to the shear stress generated by the agitation and aeration systems as well as the cell damage caused by the systems, according to Cartwright, T. “Animal Cells as Bioreactors”. New York: Cambridge University Press, 1994. 184 p.

Therefore, in order to fulfill this technological demand, it is proposed herein a bioreactor based in the concept of Taylor vortexes flow for cell culture, with efficient heat and mass transfer and low shear stress. The said bioreactor is composed basically of two concentric cylinders, wherein the internal one is rotating and the external one is stationary. From the rotation of the internal cylinder above a critical value, the formation of toroidal vortexes overlapping the main flux is initiated and they fill up the whole annular space between the two cylinders. This bioreactor is described and claimed in the present application.

### SUMMARY OF THE INVENTION

In a broad aspect, the invention relates to a Taylor vortexes flow bioreactor (TVFB), said bioreactor comprising:
a) an internal rotating body in an essentially cylindrical shape composed of external wall and fixed to a hollow tubular shaft for the flow of gases to be absorbed by the culture media;
b) in a concentric manner in relation to said internal body, an external stationary body in an essentially cylindrical shape, composed of internal wall separated from the external wall of the internal rotating body, in order to define an annular space to be filled by the culture media that contains the cells under culture, wherein the inferior part of said external body comprises a heat exchanger and lateral tubular receptacles in order to enable the introduction of electrodes (pH and dissolved oxygen) and a small duct for sample collection;
c) a dense polymeric tubular membrane highly permeable to gases such as the ones made of silicone, wherein said membrane is wrapped around the whole said internal body, which enables an efficient transfer by diffusion of the gases present in the interior of the membrane to the culture media;
d) superior lid fixed to said internal and external bodies, wherein the said lid presents holes in order to introduce the solutions and for the release of the gases present in the superior internal space of the bioreactor;
e) inferior lid;
f) mechanic seal and bearing fixed in the superior part of said superior lid, wherein the bearing presents holes in order to enable the entry of gases; and
g) agitation device responsible for spinning of said internal body through a magnetic trigger and composed by a disk in the base of the internal cylinder containing permanent magnets, while other similar disc is localized externally on a metal structure, also with permanent magnets, but of opposed polarity, exerting an attraction force, wherein said external disc is triggered by the action of electrical engine, in a way that when the rotation of said internal body surpass a critical value, the formation of toroidal vortexes overlapping the main flow is initiated and fill up all the annular space between both the internal and external bodies, wherein
h) the geometric relations between the rays of internal rᵢₙₜ and external rₑₓₜ cylinders and the aspect ratio L/d, not being limited to those, are as follow:
   Ratio between radii:

$$η = \frac{{r}_{int}}{{r}_{ext}}$$

Aspect ratio:

$$Γ = \frac{L}{d}$$

Wherein the ratio between rays (η) ranges typically from 0.1 to 0.99 and the aspect ratio (Γ) ranges typically from 0.5 to 100.

Air, oxygen, carbon dioxide, nitrogen or any mixture of gases are injected in the bioreactor through the holes present in the bearing, pass throughout the whole hollow tubular axis and diffuse from the interior of the tubular membrane to the culture media.

The invention provides a bioreactor of Taylor vortexes flow that comprises, basically: external cylinder, internal cylinder, tubular membrane, heat exchanger and a set consisting of mechanic seal and bearing, located at the top of the superior lid.

The invention also provides a bioreactor of Taylor vortexes flow comprising efficient devices for heat and mass transfer, presenting low shear stress.

The invention also provides a bioreactor of Taylor vortexes flow presenting the absence of bubble-bursting in the gas-liquid interface due to the use of dense polymeric tubular membrane that is highly permeable to gases, such as the ones made of silicone.

The invention also provides a bioreactor of Taylor vortexes flow of easy scaling up of the aeration system through the use of longer tubular membranes.

The invention additionally provides a bioreactor of Taylor vortexes flow, wherein the scaling up depends on the maintenance of the geometric relations that enables the vortexes formation.

The invention also provides a bioreactor of Taylor vortexes flow favorable to the culture of animal cells, but not limited to those, for cells in suspension as well as for cells anchored to microcarriers.

### BRIEF DESCRIPTION OF THE FIGURES

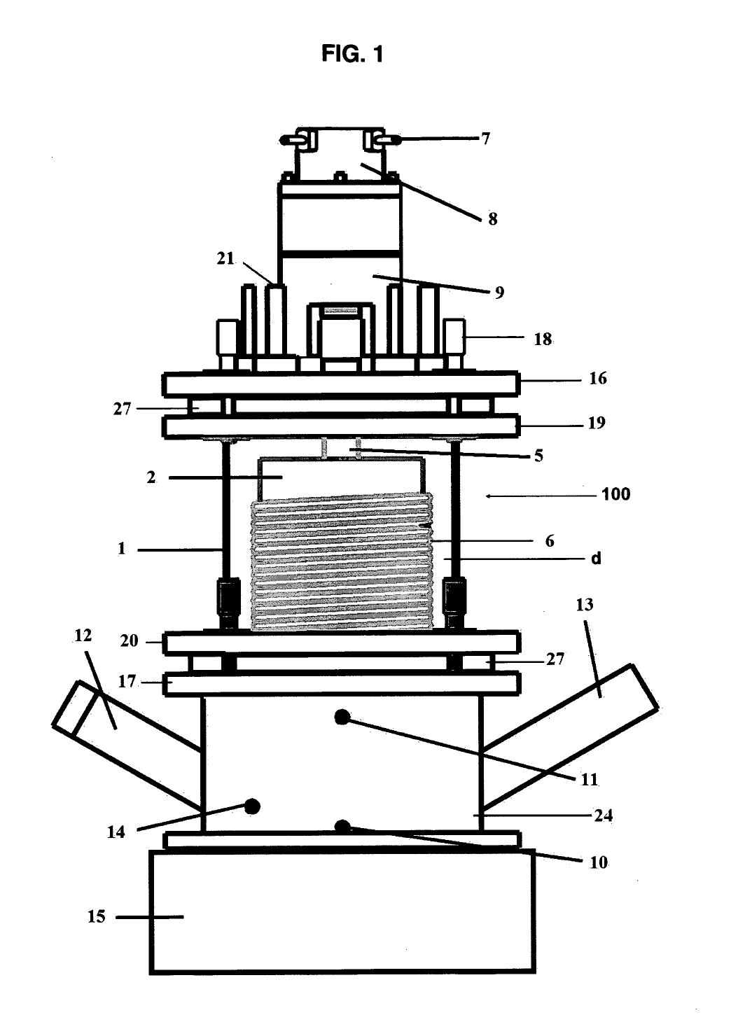

The attached FIG. 1 is a schematic representation of the bioreactor, the object of the invention.

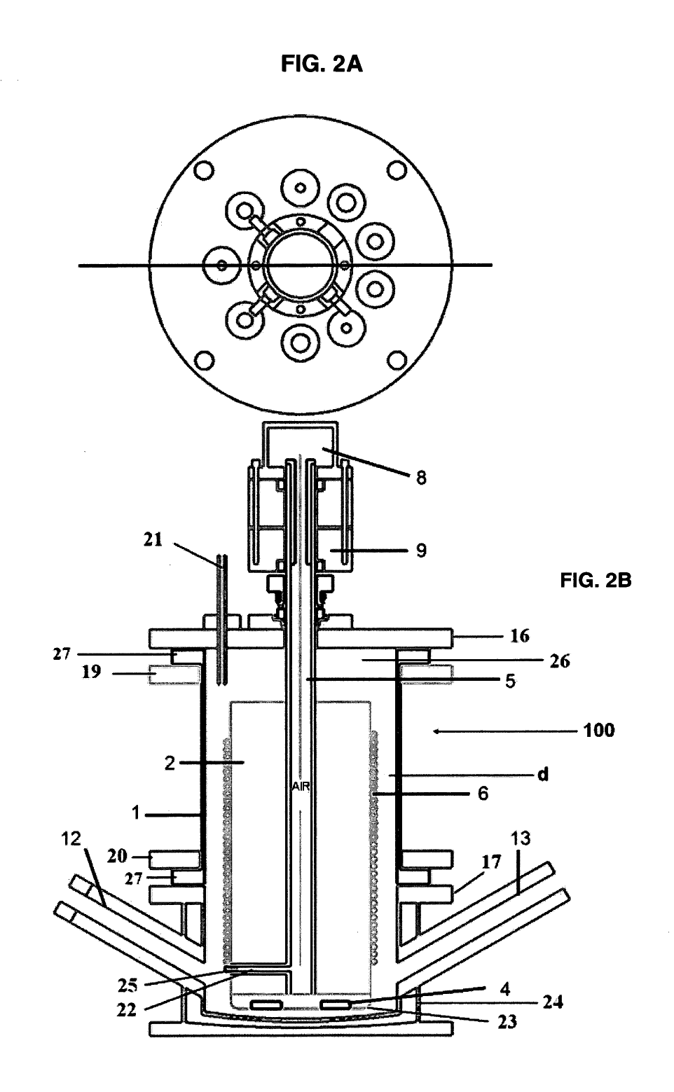

The attached FIG. 2 is a schematic drawing of a frontal section of the bioreactor of the invention. FIG. 2A illustrates the superior lid of the bioreactor. FIG. 2B is the section of the bioreactor itself.

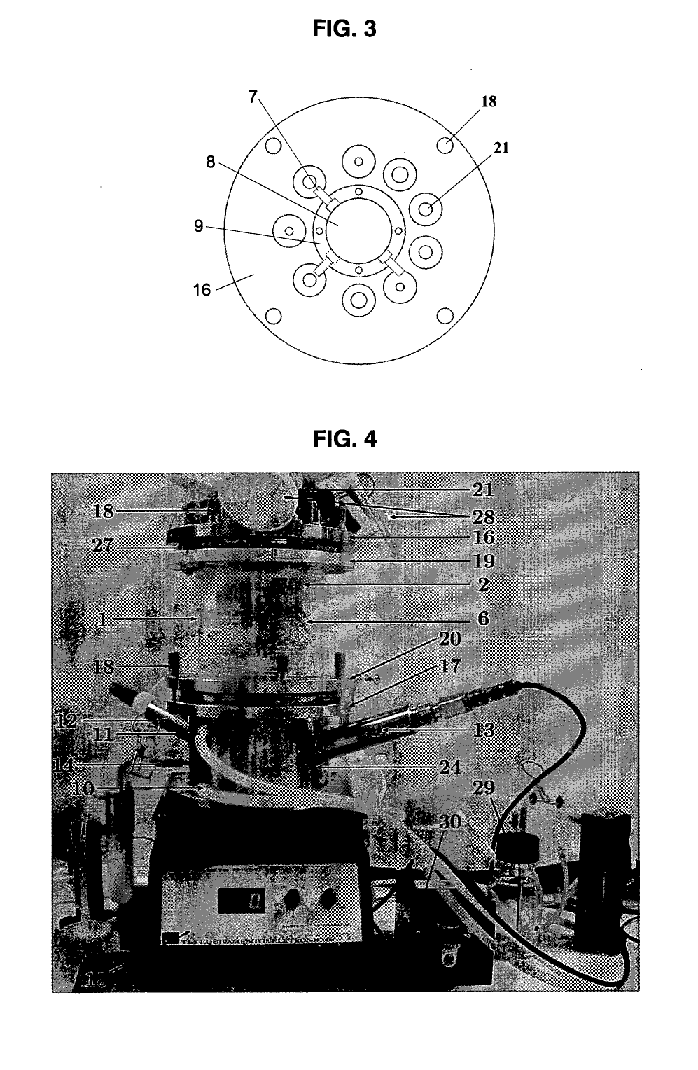

The attached FIG. 3 is a schematic reproduction of the superior lid of the bioreactor of the invention presenting holes, bearing and mechanical seal.

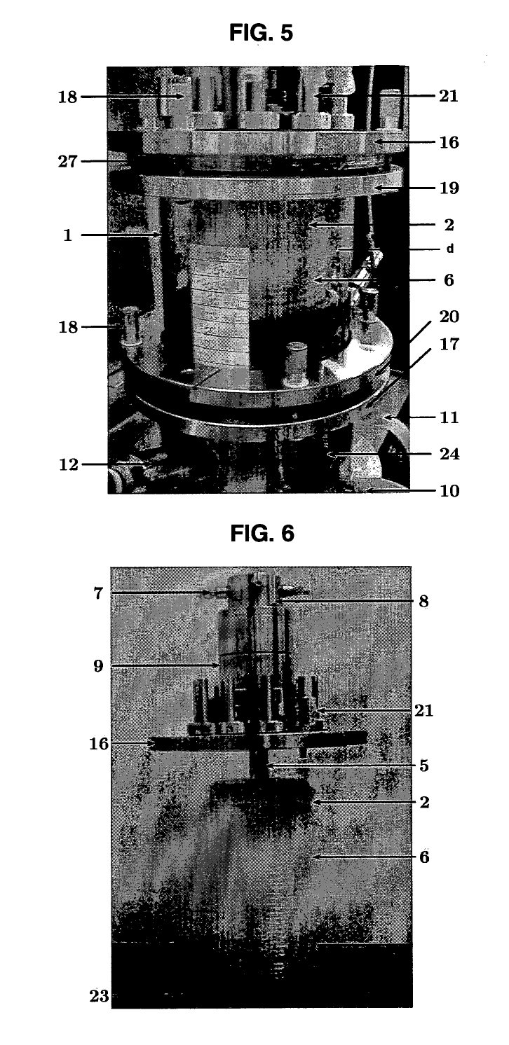

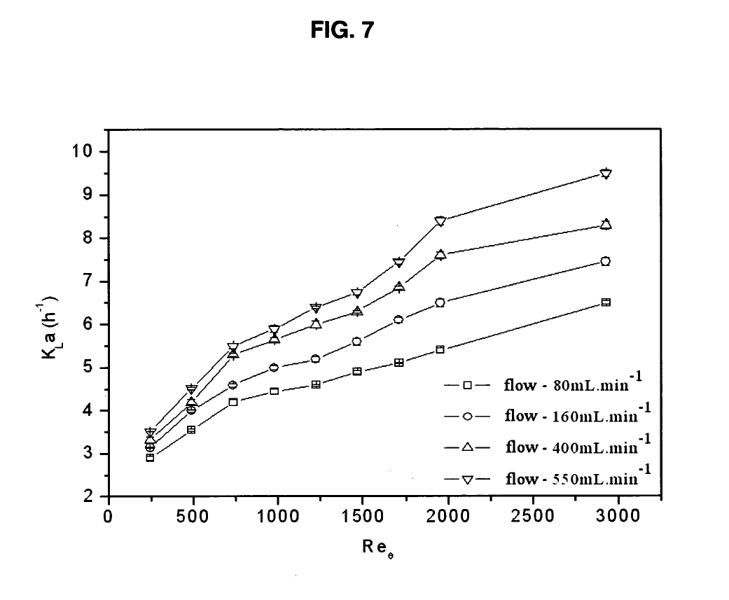

The attached FIG. 4 presents the experimental results of the global volumetric coefficient values of oxygen transfer (K_{L}a), obtained in TVFB, according to rotational Reynolds number and in different rates air of flow in the interior of the tubular membrane. The error bars corresponds to the standard deviation of the experiments performed in triplicates.

ATTACHMENT 1 is a photograph of the bioreactor, object of the invention. The bioreactor is composed of two cylindrical concentric bodies. The cylindrical body or external cylinder remains stationary while the internal one is rotating. Below the TVFB, it is found the mechanical trigger with a system that controls the speed of the rotation of the internal cylinder.

ATTACHMENT 2 is another photograph of the BTVF.

ATTACHMENT 3 is a photograph of the invention highlighting the internal rotating cylinder, the superior lid of the bioreactor and a tubular membrane around the said internal cylinder.

### DETAILED DESCRIPTION OF THE INVENTION

The Taylor vortexes flow is appropriate principally for the culture involving shear-sensitive cells, such as animal and plant cells, once the transition from the Couette flow to Taylor flow generates as global effect the reduction of shear. This reduction of the shear stress (tangential) is due to the decomposition, by the vortex, of the tension applied by the internal cylinder in the three components: axial, radial and tangential.

This condition provide a well defined flow pattern with appropriate mixing of the culture media, ensuring favorable conditions of pH, dissolved oxygen, temperature and nutrients to the cells. This is a different fact from those observed with other employed systems for cell culture, such as Spinner-type flask, roller bottles and conventional bioreactors, such as the agitating tank type.

The models of Taylor vortexes flow are based on the Taylor number (Ta) or rotational Reynolds number (Re_{θ}). Both numbers are non-dimensional and reflect the same information content about the fluid-dynamic condition of the system, which consists in the ratio between the centrifuge and viscous forces.

During the research of the Applicant that led to the results that compose the present application, the rotational Reynolds number (Re_{θ}) was selected, according to equation 1.

$${Re}_{θ} = \frac{ω {r}_{int} d}{v} \qquad ( 1 )$$

wherein, ω is the rotation speed of the internal cylinder; rᵢₙₜ is the radius of the internal cylinder; d is the annular space between the two cylinders and ν is the kinematic viscosity of the fluid in question. Wherein, Re_{θ} is at least 90.

The bioproducts to be obtained with the aid of the present bioreactor are those produced from the culture of cells, such as recombinant proteins, monoclonal antibodies, viral vaccines, biochemicals and products obtained from nucleic acids, as well as the cells themselves, which is the typical case of stem cells expansion.

One aspect of the invention is a bioreactor of Taylor vortexes flow for cell culture.

The device, object of the present invention, denominated as bioreactor of Taylor vortexes flow (TVFB), resulted from the researches of the applicant destined to supply the current need of appropriate bioreactors for cellular cultivation. The main characteristics of the TVFB are the efficient heat and mass transfers associated to low shear stress. Such characteristics have as their purpose to provide high cellular density and, consequently, increased productivity of the desired product.

The TVFB present unconventional configuration when compared to other conventional bioreactors, such as the agitating tank type and the ones with pneumatic agitation (airlift and column of bubbles).

The TVFB is composed of two concentric bodies in an essentially cylindrical shape, wherein the internal one is rotating and the external one is stationary. From the rotation of internal cylinder above a determined critical value, the formation of toroidal vortexes overlapping the main flux is initiated and they fill up the whole annular space between the two cylinders. The vortexes flow is determined by the rotation speed of the internal body, through the ratio between the cylinders radii and by the kinematic viscosity of the media.

The internal rotating body is composed of external wall and it is fixed to a hollow tubular axis for delivery of gases absorbed by the culture media. The external stationary body is composed of internal wall separated from the external wall of the rotating body, in order to define an annular space to be occupied in part by the tubular membrane and in part by the culture media containing the cells.

Advantageously, the invention uses a dense polymeric tubular membrane that is highly permeable to gases, located around the whole internal cylindrical body. The objective of the employment of this membrane is the supplement, by diffusion to the culture media, of gases necessary to the cultivation of cells and, therefore, avoiding the cellular destruction due to the bubble-bursting in the gas-liquid interface of the bioreactor.

The bioreactor additionally comprises a device that enables the spinning of the said internal body through magnetic trigger, since the said internal body presents permanent magnets in its base, while another similar disk, also presenting permanent magnets, with polarities opposed to the ones of the said internal body, located externally in a metal structure, is triggered by the action of an electrical engine that enables the control of the rotation speed of the said internal body.

The invention is described as follow through reference to Figures and Attachments. However, it will become evident to those skilled in the art that many modifications and variations are possible from the methods present within the scope of the invention.

In FIG. 1, it is presented the schematic drawing of the bioreactor of the invention, denominated as Taylor vortexes flow bioreactor (TVFB). The bioreactor is designated, in general, by the numeral (100).

In the embodiment present, the TVFB was designed with an usable volume of 1.0 L, wherein the said volume is defined by the annular space (d) between the two concentric cylinders (1) and (2). The external cylinder (1) is constituted, in the superior part, of a tank made of borosilicate glass and, in the inferior part, of a heat exchanger (24) made of stainless steel material, such as stainless steel 316L, not being limited to that. The function of the heat exchanger (24) is to keep temperature, inside the bioreactor (100), in the value selected for the cultivation that is intended. For this, water derived from an external thermostatic bath (not shown in the Figure) is employed to circulate in the heat exchanger (24). The liquid path can be visualized in FIG. 1, wherein the entrance (10) and exit (11) of the heat exchanger (24) are through the holes present in the said bioreactor (100).

In the tubular receptacles fixed to the vertical wall of the inferior metallic body (24) of the bioreactor, the electrodes of pH (12) and dissolved oxygen (13) are introduced. A small duct (14), also fixed to this wall, is used for sample collection. The electrodes ((12) and (13)) are coupled to commercially available external measurers/transmitters (not shown in the Figure).

The internal cylinder (2) is made of a polymer that is resistant to high temperatures, such as polypropylene, not being limited to that, and fixed to a hollow tubular axis (5) made of non oxidizing material, such as stainless steel 316L, not being limited to that.

Around the whole internal cylinder (2), it is found a wrapped-around dense polymeric tubular membrane (6) that is highly permeable to gases, such as air, carbon dioxide, oxygen, nitrogen or mixture of any gases. The gases (air, carbon dioxide, oxygen, nitrogen or mixture of any gases) path in the interior of the TVFB (100) can be followed in FIG. 2.

The gases introduced in the bioreactor (100) through the holes (7) present in the bearing (8), flow through the interior of the said tubular shaft (5), are released at the base (22) of the internal cylinder (2) and, through a connection (25), pass to the said tubular membrane (6). The gas transfer to the interior of the bioreactor and, consequently, to the culture media, is due to the diffusion mechanism through the wall of the said polymeric tubular membrane (6).

This system, besides enabling the provision of gases to the culture media, avoids the occurrence and, consequently, the bursting of air bubbles in the gas-liquid interface.

After circulating throughout the tubular membrane (6), part of the gases is released in the superior internal space (26) of the bioreactor (100). At this location, the gases are released to the external environment through filters that can be sterilized (28) (see Attachment 1), installed at the holes (21) present in the superior lid (16) thereof.

In FIG. 2, it is possible to visualize the agitation system of the bioreactor (100). The internal cylinder (2) is propelled by an magnetic trigger of the disk (23) located at its base, made of non oxidizing material, such as stainless steel 316L, not being limited to that, and containing in its interior permanent magnets (4).

Other similar disk is located externally in the metal structure (15) (see FIG. 1) also presenting permanent magnets, but of opposed polarity, exerting an attraction force, and it is triggered by the action of an electrical engine (present in the metal structure (15) and not shown in the Figure), enabling the control of the rotation speed of the internal cylinder (2).

FIG. 3 illustrates the schematic drawing of the superior lid (16) of the bioreactor (100). The lid is made of non oxidizing material, such as stainless steel, not being limited to that, and present openings (21) that are used for addition of solutions, such as base, acid, inocule and culture media, in the interior of the bioreactor (100). These solutions, kept in appropriate containers (29) for this purpose (see Attachment 1), are inserted in the bioreactor (100) with the aid of a peristaltic pump (30) (see Attachment 1). In the superior part of the lid (16) the mechanical seal (9) and the bearing (8) are fixed. In the latter, the openings allow the entrance of gases (7) (air, carbon dioxide, oxygen, nitrogen or a mixture of any of the gases) in the interior of the said bioreactor ad shown in FIG. 2.

The attachments 1, 2 and 3 are photographs of the “Taylor Vortexes Flow Bioreactor” (TVFB) and details thereof.

On Table 1 geometric characteristics of the TVFB are mentioned as example, not being necessarily limited to those.

TABLE 1
Geometric characteristics
$\text{Ratio between radii:} η = \frac{{r}_{int}}{{r}_{ext}}$
$\text{Aspect ratio:} Γ = \frac{L}{d}$

wherein, rᵢₙₜ corresponds to the radius of the internal cylinder, rₑₓₜ is the radius of the external cylinder (1), L corresponds to the axial length of the internal cylinder (2) and d corresponds to the annular space between the internal cylinder (2) and the external cylinder (1). The ratio between the rays (η) can range from 0.1 to 0.99 and the aspect ratio (Γ), for example, from 0.5 to 100, with typical values between 0.3 and 0.90 for the ratio between rays (η) and between 10 and 60 for the aspect ratio (Γ).

After the conclusion of the steps of the project and construction of the bioreactor, the transfer of heat and oxygen were evaluated inside it.

For 10 days (240 h) the bioreactor remained on and in its interior, sterile culture media DMEM (Dulbeco's Modified Eagle's Medium) was added. During the experiments, the selected rotation speeds of the internal cylinder, 50, 100 and 200 rpm, were kept constant. It could be observed with the results that the selected temperature (37° C.) was kept constant and that the culture media remained sterile through the daily sample collections.

The bioreactor (100) present appropriate oxygen transfer capacity to the reaction media. This characteristic is a consequence of the formation of the Taylor vortexes flow and the use of a tubular membrane (6), around the whole internal cylinder (2).

The determination of the global volumetric coefficient of oxygen transfer (K_{L}a) in the TVFB was based on the dynamic method, which uses the response signal of the oxygen electrode immersed in the liquid submitted to aeration, according to Blanch, H. W.; Clark, D. S. “Biochemical Engineering” New York: M. Dekker Inc., 1997. Cap.5, p. 343-452.

In the TVFB, the determination of K_{L}a consisted of experiments performed in the absence of cells and in different agitation conditions (rotation speed of the internal cylinder) and aeration (rate of air flow in the interior of the silicone tubular membrane).

In a typical example, the bioreactor (100) was operated with 800 mL of DMEM culture media at a temperature of 37° C. The operational conditions were: rotation speeds of the internal cylinder ranging between 25 and 300 rpm and rates of air flow ranging between 80 and 550 mL·min⁻¹.

When analyzing the data presented in the graph of FIG. 4, it can be verified that the Taylor vortexes flow regime increases the transport of oxygen when compared to other bioreactors that also employ this type of membrane.

As example of comparison, experiments performed with bioreactor of the agitating tank type, operated at 80 rpm and tubular membrane of 50 m, (external diameter of 3.2 mm and wall thickness of 0.6 mm), resulted in K_{L}a values between 2 and 3 h⁻¹, as published in Tonso, A. “Monitoramento e operação de cultivos de células animais em sistemas de perfusão” Dissertation (PhD in Chemical Engineering)—Departamento de Engenharia Química, Escola Politécnica da Universidade de São Paulo, São Paulo. 2000. Similar K_{L}a value were presented in Qi, H. N. et al. “Experimental and theoretical analysis of tubular membrane aeration for mammalian cell bioreactors”. Biotechnology Progress, v. 19, p. 1183-1189. 2003. In that work, the extension of the tubular membrane was of 80 m and the wall thickness of the tubular membrane was of 0.55 mm.

In the experiment performed with the TVFB, operated in conditions similar to the ones of the bioreactors of agitating tank type, Kₗa value was of 5.5 h⁻¹. The advantage of the invention was to enable the reduction in the length of the tubular membrane to only 7.5 m.

Another aspect of the invention is its functioning.

Basically, the functioning of the bioreactor of the invention involves its closing followed by the introduction of the cells (inoculum) through the appropriate entrance, besides the base and acid solutions for pH control, and also the gases that are diffused to the culture media with the aid of tubular membrane, which provides improved oxygen transfer inside the bioreactor.

The bioprocess is initiated by triggering the bioreactor through the rotation of the internal cylinder, in such way that when this rotation surpasses a critical value, the formation of toroidal vortexes overlapping the main flux is initiated and fills up the whole annular space between the two cylinders, favoring the cultivation of cells under low shear stress. After the rotation of the engine is stopped, the cultivation of cells is recovered.

First, the bioreactor (100) must be correctly closed (or sealed) before being autoclaved. This procedure of closing is as follows: the external glass cylinder (1) is composed, in its ends, of flanges (27). The lids, superior (16) and inferior (17), present furrows where the sealing rings are placed (not shown in the Figure) that can be autoclaved and are made of viton or similar material, not being limited to that. For the closing of the lids (16) and (17), the sealing rings are placed in the flanges (27) and four screws nuts and threaded (18) are employed to unite the said lids (16) and (17) to the fixation rings (19) and (20), made of non oxidizing material, such as aluminum and stainless steel, pressing this way the flanges (27).

After the previous steps, culture media, cells (inoculum), base, acid are introduced through the holes (21) in the bioreactor (100) through the use of a peristaltic pump (30). The delivery of gases is through the holes (7) located in the superior part of the bearing (8). The gases flow via hollow tubular shaft (5), are released at the base (22) of the internal cylinder (2) and, through a connection (25), pass to the tubular membrane (6). The tubular membrane (6) enables the provision of gases to the culture media by diffusion without the occurrence of air bubbles. The gases, after circulating throughout the whole tubular membrane (6), are released in the superior part (26) of the bioreactor. At this location, the gases are released to the external environment through filters that can be sterilized (28), present at the holes (21) present in the superior lid (16) thereof. The temperature in the interior of the bioreactor is kept in the appropriate range with the aid of a heat exchanger (24).

In order to turn the bioprocess on, the agitation system of the bioreactor (100) is triggered. This mechanism is generated by the rotation of the internal cylinder (2), propelled by the magnetic trigger of the disk (23) containing in its interior four permanent magnets (4), while other similar disk, also containing permanent magnets, but with opposed polarities, located externally in the metal structure (15), is triggered by an electrical engine. From the rotation of the internal cylinder (2) above a critical value, the formation of toroidal vortexes overlapping the main flux is initiates and fills up the whole annular space (d) between the two cylinders. The vortexes flow is determined by the rotation speed of the internal cylinder (2), through the ratio between the rays of the cylinders and through the kinematic viscosity of the media. During the experiments, samples are collected through the opening (14) present in the inferior part of the external cylinder (1). After the reaction is finished, there is the interruption of the engine rotation, the bioreactor is opened and the products are collects and appropriately stored.

The bioreactor (100) besides presenting a novel conception based in the concept of Taylor vortexes flow, presents the advantages of low cost, easy construction and scaling up, and efficient mechanisms of heat (due to the heat exchanger located in the inferior part of the equipment) and mass transfer.

The bioreactor of the invention also presents the possibility of installation of peripherical devices to monitor and control the parameters of the culture of cells.

The present bioreactor employs the Taylor vortexes flow for the culture of cells in suspension as well as the ones dependent of anchoring to microcarriers.

The innovation of the bioreactor consists of the development of a device that enables the scaling up of the oxygenation system, increasing considerately the oxygen transfer inside the equipment, when compared to other bioreactors of Taylor vortexes flow that do not employ this conception. In this device, a tubular membrane is installed around the whole internal cylinder. This way, the geometry of the membrane-culture media contact area is more favorable, allowing the increase of the ratio transfer area by reactor volume, simply increasing the extension of the tubular membrane. The efficiency of this conception was extensively proved by experiments presented in FIG. 4 that compose the present report.

Other advantages of the invention regarding the state of the technique are the low shear stress generated in the fluid-dynamic environment and the absence of bubble-bursting in the gas-liquid interface due to the presence of the tubular membrane.

Besides that, there is the scaling up easiness of the bioreactor, as long as the geometric relations are maintained which enable the formation of the vortexes flow.

That which is claimed:

1. Taylor vortex flow bioreactor for cell culture, wherein said bioreactor comprising:
a) an internal rotating body (2) in an essentially cylindrical shape composed of external wall and fixed to a hollow tubular shaft (5) for the flow of gases to be absorbed by the culture media;
b) in a concentric manner in relation to said internal body (2), an external stationary body (1) in an essentially cylindrical shape, composed of internal wall separated from the external wall of the internal rotating body (2), in order to define an annular space (d) to be filled by the culture media that contains the cells under cultivation, wherein the inferior part of said external body is rested over a cylindrical base, made of stainless steel which acts as a heat exchanger (24) in order to enable the temperature modulation of the culture media;
c) the inferior part of said external body being constituted of lateral tubular receptacle in order to enable the introduction of pH electrodes (12) and dissolved oxygen (13) and a small duct (14) for sample collection;
d) a dense polymeric tubular membrane (6) highly permeable to gases wherein said membrane (6) is wrapped around the whole said internal body (2), which enables an efficient transfer by diffusion of the gases present in the interior of the membrane to the culture media;
e) superior lid (16) supporting the said internal body (2) and leaning over the external (1) body, wherein the said lid presents holes (21) in order to introduce the solutions and for the release of the gases present in the superior internal space (26) of the bioreactor;
f) inferior lid (17);
g) mechanical seal (9) and bearing (8) fixed in the superior part of said superior lid (16), wherein the shaft (5) crossing aseptically both the bearing housing (8) and the lid (16) is hollow in order to enable the entry of gases; and
h) agitation device responsible for spinning of said internal body (2) through a magnetic trigger and composed by a disk (23) in the base of the internal cylinder (2) containing permanent magnets (4), while other similar disc is localized externally on a metal structure (15), also with permanent magnets, but of opposed polarity, exerting an attraction force, wherein said external disc is triggered by the action of electrical engine, in a way that when the rotation of said internal body (2) surpass a critical value, the formation of toroidal vortexes overlapping the main flow is initiated and fill up all the annular space (d) between both the internal (2) and external (1) bodies, wherein the geometric relations between the radii of internal (rᵢₙₜ) and external (rₑₓₜ) cylinders and the aspect ratio L/d, are:
Ratio between rays:
$$η = \frac{{r}_{int}}{{r}_{ext}}$$
typically ranging from 0.1 to 0.99 and
Aspect ratio:
$$Γ = \frac{L}{d}$$
typically ranging from 0.5 to 100.

2. Bioreactor according to claim 1, wherein the cellular cultivation has as objective to obtain bioproducts as recombinant proteins, monoclonal antibodies, viral vaccines, biochemicals and products obtained from nucleic acids, as well as the cells themselves, as is the typical case of expansion of stem cells.

3. Bioreactor according to claim 2, wherein the cells are in suspension.

4. Bioreactor according to claim 2, wherein the cells are anchored to microcarries.

5. Bioreactor according to claim 1, wherein the ratio between the radii is between 0.3 and 0.90 and the aspect ratio between 10 and 60.

6. Bioreactor according to claim 1, wherein the scaling up of the aeration system thereof consists in the alteration of the tubular membrane (6) extension, while the geometric relations between the radii of internal rᵢₙₜ and external rₑₓₜ cylinders and the aspect ratio L are maintained.

7. Bioreactor according claim 1, wherein the rotational Reynolds number (Re_{θ}) is at least 90.

8. Bioreactor according to claim 1, wherein while functioning, it operate according to the follow steps:
a) to seal said bioreactor in order to enable operation in an aseptic manner, joining the lids (16) and (17) to the rings (19) and (20) and tightening the flanges (27);
b) to introduce, by using a peristaltic pump (30), through the holes (21), culture media, cells (inoculum) and basic and acidic solutions;
c) to introduce gases through the holes (7) localized in the superior part of the bearing housing (8) toward the hollow tubular shaft (5), up to the release of those in the base (22) of the internal cylinder (2) and flow of those to the tubular membrane (6) through the connection (25), transfer of gases to the culture media by diffusion, circulating through in the interior of said tubular membrane (6), wherein they are released in the superior part (26) of the bioreactor and leaving this through filters that can be sterilized (28) present in the holes (21) of the superior lid (16);
d) to maintain the temperature of the interior of the bioreactor in the appropriate range with the aid of the heater exchanger (24);
e) to trigger the agitation system generated by the rotation of the internal cylinder (2), propelled by the magnetic trigger of two discs (23) of opposed polarity containing permanent magnets (4), in a way that, from the rotation of said internal cylinder (2) above an critical value the formation of toroidal vortexes overlapping the main flow is initiated and fill up the whole annular space (d) between the internal (2) and external (1) cylinders;
f) to maintain, through rotation of the internal cylinders (2), low shear stress and a good homogenization of the culture media, enabling that nutrients, dissolved oxygen and temperature be equally distributed to the cells, in order to enable the evolution of the bioprocess with high efficiency and high yield;
g) to collect samples during the experiments through the opening (14) located in the inferior part of the external cylinder (1);
h) to interrupt the rotation of the engine, located in the interior of the metal structure (15), after the conclusion of the reaction and to open the bioreactor in an aseptic manner; and
i) to collect and to store the products of the bioprocess.
